(19) United States
(12) Patent Application Publication
(10) Pub. No.: US 2024/0300988 A1
(43) Pub. Date: Sep. 12, 2024

(54) CRYSTALLINE FORMS OF PENTAAZA MACROCYCLIC RING COMPLEX

(71) Applicant: Galera Labs, LLC, Malvern, PA (US)

(72) Inventors: Stephan D. PARENT, West Lafayette, IN (US); Tanise SHATTOCK-GORDON, West Lafayette, IN (US)

(21) Appl. No.: 18/598,251

(22) Filed: Mar. 7, 2024

Related U.S. Application Data
(60) Provisional application No. 63/451,107, filed on Mar. 9, 2023.

(51) Int. Cl. C07F 13/00; A61K 31/555

(52) U.S. Cl. CPC C07F 13/005; A61K 31/555; C07B 2200/13

(57) ABSTRACT

The present disclosure provides polymorph forms of pentaaza macrocyclic ring

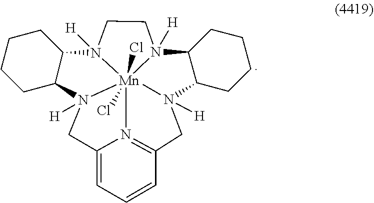

Also provided are pharmaceutical compositions that include the provided polymorph forms and methods of using the provided polymorph forms and pharmaceutical compositions for the treatment of disease states.

### CROSS-REFERENCE TO RELATED APPLICATIONS

The present application claims benefit of priority to U.S. Provisional Patent Application No. 63/451,107, filed Mar. 9, 2023, the entire content of which is hereby incorporated by reference herein in its entirety, as if recited in full herein.

### FIELD OF THE DISCLOSURE

The present disclosure relates to crystalline forms of a pentaaza macrocyclic ring complex, which are useful in the treatment of various cancers and inflammatory disorders such as oral mucositis, among other conditions.

### BACKGROUND OF THE DISCLOSURE

Transition metal-containing pentaaza macrocyclic ring complexes having the macrocyclic ring system corresponding to the formula below have been shown to be effective in a number of animal and cell models of human disease, as well as in treatment of conditions afflicting human patients.

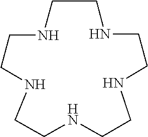 [SMILES: C1CNCCNCCNCCNCCN1]

For example, one such compound, GC4419, has been shown to attenuate VEGFr inhibitor-induced pulmonary disease in a rat model (Tuder, et al., Am. J. Respir. Cell Mol. Biol., 29, 88-97 (2003)).

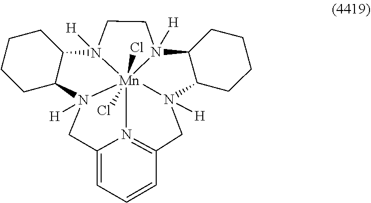

The mirror image of GC4419 is yet another one of these compounds, the chemical structure of which (GC4403) is shown below:

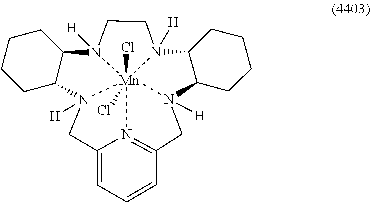

Pharmaceutical compositions are often formulated with a crystalline solid of the active pharmaceutical ingredient (API). The specific crystalline form of the API can have significant effects on properties such as stability and solubility/bioavailability. Instability and solubility characteristics can limit the ability to formulate a composition with an adequate shelf life or to effectively deliver a desired amount of a drug over a given time frame.

There exists an unmet need for crystalline forms of GC4419 and GC4403, which exhibit improved properties for formulation of pharmaceutical compositions. The present disclosure is directed to meeting this and other needs.

### SUMMARY OF THE DISCLOSURE

The present disclosure is directed to various crystalline forms of GC4419. Such solid forms are referred to herein as Form V, Form VI, Form VIII, and Form XI. The forms are either hydrates (Form V) or anhydrates (Form VI, Form VIII, and Form XI).

Furthermore, corresponding crystalline forms are also provided for GC4403 (also referred to as M40403), which is the mirror image form of GC4419, and differs only in its chirality from GC4419, but does not otherwise differ in terms of chemical structure. Thus, for each crystalline form of GC4419, there is a corresponding crystalline form of GC4403, and would be expected to be obtainable by the same or substantially similar process described herein for GC4419.

Accordingly, the present disclosure provides crystalline forms of GC4419 and GC4403.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the chemical structure of GC4419.

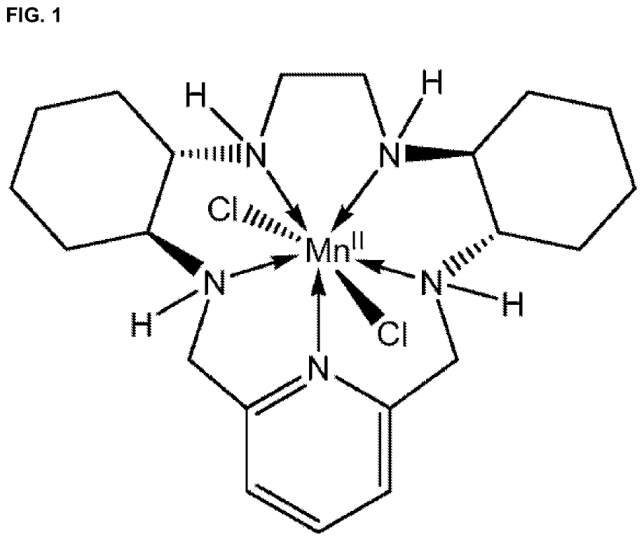

FIG. 2 shows an XRPD pattern of lots of GC4419 used to prepare the crystalline forms of the disclosure.

FIG. 3 shows an XRPD pattern of Form V.

FIG. 4 shows thermograms of Form V.

FIG. 5 shows an XRPD pattern comparison upon drying of Form V over P₂O₅.

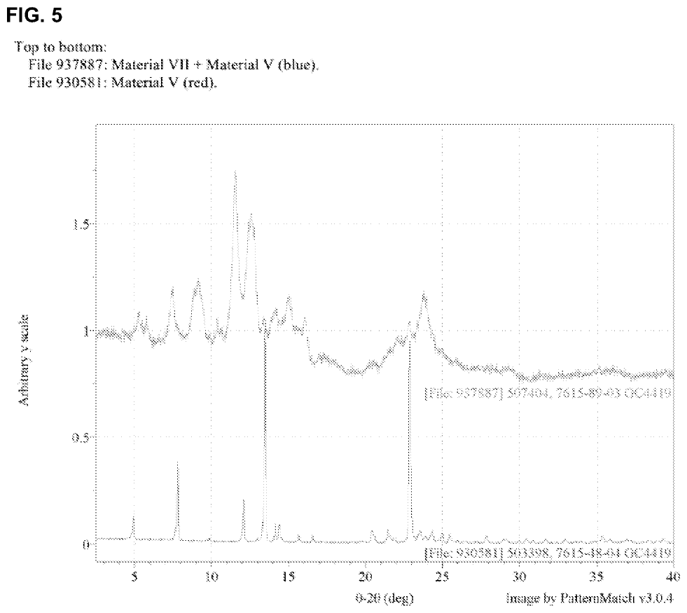

FIG. 6 shows an XRPD pattern comparison of Form VIII with a monohydrate of GC4419.

FIG. 7 shows a tentative indexing result for Form VIII.

FIG. 8 shows an XRPD pattern comparison of Form XI and Form VIII.

FIG. 9 shows an XRPD pattern containing Form VI (plus an extra peak).

### DEFINITIONS

The term “solid form” is often used to refer to a class or type of solid-state material. One kind of solid form is a “polymorph” which refers to two or more compounds having the same chemical formula but differing in solid-state structure. Salts may be polymorphic. When polymorphs are elements, they are termed allotropes. Carbon possesses the well-known allotropes of graphite, diamond, and buckminsterfullerene. Polymorphs of molecular compounds, such as active pharmaceutical ingredients (“APIs”), are often prepared and studied in order to identify compounds meeting scientific or commercial needs including, but not limited to, improved solubility, dissolution rate, hygroscopicity, and stability.

Other solid forms include solvates and hydrates of compounds including salts. A solvate is a compound wherein a solvent molecule is present in the crystal structure together with another compound, such as an API. When the solvent is water, the solvent is termed a hydrate. Solvates and hydrates may be stoichiometric or non-stoichiometric. A monohydrate is the term used when there is one water molecule, stoichiometrically, with respect to, for example, an API, in the unit cell.

In order to identify the presence of a particular solid form, one of ordinary skill typically uses a suitable analytical technique to collect data on the form for analysis. For example, chemical identity of solid forms can often be determined with solution-state techniques such as ¹³C-NMR or ¹H-NMR spectroscopy and such techniques may also be valuable in determining the stoichiometry and presence of “guests” such as water or solvent in a hydrate or solvate, respectively. These spectroscopic techniques may also be used to distinguish, for example, solid forms without water or solvent in the unit cell (often referred to as “anhydrates”), from hydrates or solvates.

Solution-state analytical techniques do not provide information about the solid state as a substance and thus, for example, solid-state techniques may be used to distinguish among solid forms such as anhydrates. Examples of solid-state techniques which may be used to analyze and characterize solid forms, including anhydrates and hydrates, include single crystal X-ray diffraction, X-ray powder diffraction (“XRPD”), solid-state ¹³C-NMR, Infrared (“IR”) spectroscopy, including Fourier Transform Infrared (FT-IR) spectroscopy, Raman spectroscopy, and thermal techniques such as Differential Scanning calorimetry (DSC), melting point, and hot stage microscopy.

Polymorphs are a subset of crystalline forms that share the same chemical structure but differ in how the molecules are packed in a solid. When attempting to distinguish polymorphs based on analytical data, one looks for data which characterize the form. For example, when there are two polymorphs of a compound (e.g., Form I and Form II), one can use X-ray powder diffraction peaks to characterize the forms when one finds a peak in a Form I pattern at angles where no such peak is present in the Form II pattern. In such a case, that single peak for Form I distinguishes it from Form II and may further act to characterize Form I. When more forms are present, then the same analysis is also done for the other polymorphs. Thus, to characterize Form I against the other polymorphs, one would look for peaks in Form I at angles where such peaks are not present in the X-ray powder diffraction patterns of the other polymorphs. The collection of peaks, or indeed a single peak, which distinguishes Form I from the other known polymorphs is a collection of peaks which may be used to characterize Form I. If, for example, two peaks characterize a polymorph then those two peaks can be used to identify the presence of that polymorph and hence characterize the polymorph. Those of ordinary skill in the art will recognize that there are often multiple ways, including multiple ways using the same analytical technique, to characterize polymorphic polymorphs. For example, one may find that three X-ray powder diffraction peaks characterize a polymorph. Additional peaks could also be used, but are not necessary, to characterize the polymorph up to and including an entire diffraction pattern. Although all the peaks within an entire diffractogram may be used to characterize a crystalline form, one may instead, and typically does as disclosed herein, use a subset of that data to characterize such a crystalline form depending on the circumstances.

When analyzing data to distinguish an anhydrate from a hydrate, for example, one can rely on the fact that the two solid forms have different chemical structures—one having water in the unit cell and the other not. Thus, this feature alone may be used to distinguish the forms of the compound and it may not be necessary to identify peaks in the anhydrate, for example, which are not present in the hydrate or vice versa.

X-ray powder diffraction patterns are some of the most commonly used solid-state analytical techniques used to characterize solid forms. An X-ray powder diffraction pattern is an x-y graph with the diffraction angle, 2θ(°), on the x-axis and intensity on the y-axis. The peaks within this plot may be used to characterize a crystalline solid form. The data is often represented by the position of the peaks on the x-axis rather than the intensity of peaks on the y-axis because peak intensity can be particularly sensitive to sample orientation (see Pharmaceutical Analysis, Lee & Web, pp. 255-257 (2003)). Thus, intensity is not typically used by those skilled in the art to characterize solid forms.

As with any data measurement, there is variability in X-ray powder diffraction data. In addition to the variability in peak intensity, there is also variability in the position of peaks on the x-axis. This variability can, however, typically be accounted for when reporting the positions of peaks for purposes of characterization. Such variability in the position of peaks along the x-axis derives from several sources. One comes from sample preparation. Samples of the same crystalline material, prepared under different conditions may yield slightly different diffractograms. Factors such as particle size, moisture content, solvent content, and orientation may all affect how a sample diffracts X-rays. Another source of variability comes from instrument parameters. Different X-ray instruments operate using different parameters and these may lead to slightly different diffraction patterns from the same crystalline solid form. Likewise, different software packages process X-ray data differently and this also leads to variability. These and other sources of variability are known to those of ordinary skill in the pharmaceutical arts.

Due to such sources of variability, it is common to recite X-ray diffraction peaks using the word “about” prior to the peak value in degrees (2θ) (sometimes expressed herein as “2θ-reflections (°)”), which presents the data to within 0.1 or 0.2° (2θ) of the stated peak value depending on the circumstances. The X-ray powder diffraction data corresponding to the solid forms of the present disclosure were collected on instruments which were routinely calibrated and operated by skilled scientists. In the present disclosure, XRPD values are preferably obtained using Cu Kα X-ray radiation according to the method described in Example 1. Accordingly, the variability associated with these data would be expected to be closer to ±0.1° 2θ than to ±0.2° 2θ and indeed likely less than 0.1 with the instruments used herein. However, to take into account that instruments used elsewhere by those of ordinary skill in the art may not be so maintained, for example, all X-ray powder diffraction peaks cited herein have been reported with a variability on the order of ±0.2° 2θ and are intended to be reported with such a variability whenever disclosed herein and are reported in the specification to one significant figure after the decimal even though analytical output may suggest higher precision on its face.

Single-crystal X-ray diffraction provides three-dimensional structural information about the positions of atoms and bonds in a crystal. It is not always possible or feasible, however, to obtain such a structure from a crystal, due to, for example, insufficient crystal size or difficulty in preparing crystals of sufficient quality for single-crystal X-ray diffraction.

X-ray powder diffraction data may also be used, in some circumstances, to determine the crystallographic unit cell of the crystalline structure. The method by which this is done is called “indexing.” Indexing is the process of determining the size and shape of the crystallographic unit cell consistent with the peak positions in a suitable X-ray powder diffraction pattern. Indexing provides solutions for the three unit cell lengths (a, b, c), three unit cell angles (α, β, γ), and three Miller index labels (h, k, l) for each peak. The lengths are typically reported in Angstrom units and the angles in degree units. The Miller index labels are unitless integers. Successful indexing indicates that the sample is composed of one crystalline phase and is therefore not a mixture of crystalline phases.

IR spectroscopy, particularly FT-IR, is another technique that may be used to characterize solid forms together with or separately from X-ray powder diffraction. In an IR spectrum, absorbed light is plotted on the x-axis of a graph in the units of “wavenumber” (cm⁻¹), with intensity on the y-axis. Variation in the position of IR peaks also exists and may be due to sample conditions as well as data collection and processing. The typical variability in IR spectra reported herein is on the order of plus or minus 2.0 cm⁻¹. Thus, the use of the word “about” when referencing IR peaks is meant to include this variability and all IR peaks disclosed herein are intended to be reported with such variability.

Thermal methods are another typical technique to characterize solid forms. Different polymorphs of the same compound often melt at different temperatures. Thus, the melting point of a polymorph, as measured by methods such as capillary melting point, DSC, and hot stage microscopy, alone or in combination with techniques such as X-ray powder diffraction, IR spectroscopy, including FT-IR, or both, may be used to characterize polymorphs or other solid forms.

As with any analytical technique, melting point determinations are also subject to variability. Common sources of variability, in addition to instrumental variability, are due to colligative properties such as the presence of other solid forms or other impurities within a sample whose melting point is being measured.

The following examples are provided to further illustrate the compounds, compositions and methods of the present disclosure. These examples are illustrative only and are not intended to limit the scope of the disclosure in any way.

### EXAMPLES

### Example 1

### Experimental Settings

### Indexing

Indexing is the process of determining the size and shape of the crystallographic unit cell given the peak positions in a diffraction pattern. The term gets its name from the assignment of Miller index labels to individual peaks. Agreement between the allowed peak positions, marked with red bars within the figures, and the observed peaks indicates a consistent unit cell determination. To confirm the tentative indexing solution, the molecular packing motifs within the crystallographic unit cells must be determined. No attempts at molecular packing were performed.

XRPD indexing serves several purposes. If all of the peaks in a pattern are indexed by a single unit cell, this is strong evidence that the sample contains a single crystalline phase. Given the indexing solution, the unit cell volume may be calculated directly. The difference in volume per formula unit between an unsolvated crystal form (estimated, if not known) and the volume per formula unit of hydrates and/or solvates can be useful in the determination of the potential extent of hydration/solvation. Indexing is also a robust description of a crystalline form, through tentative crystallographic unit cell parameters, and provides a concise summary of all available peak positions for that phase at a particular thermodynamic state point.

### Cooling and Slow Cools

Solutions were prepared in the selected solvent or solvent/anti-solvent system. These solutions were chilled below room temperature within a refrigerator for varying lengths of time in an attempt to induce nucleation. The presence or absence of solids was noted. Upon observation of solids, in quantities sufficient for analysis, isolation of material was conduction. If insufficient quantities were present further cooling was performed in a freezer. Samples were either isolated for analysis wet or as dry powders.

### Fast Evaporation

Solutions were prepared in selected solvents and agitated between aliquot additions to assist in dissolution. Once a mixture reached complete dissolution, as judged by visual observation, the solution was filtered through a 0.2-μm nylon filter and allowed to evaporate at ambient temperature in an uncapped vial or at ambient under nitrogen. The solids that formed were isolated for evaluation.

### Slow Evaporation

Solutions were prepared in selected solvents and agitated between aliquot additions to assist in dissolution. Once a mixture reached complete dissolution, as judged by visual observation, the solution was filtered through a 0.2-μm nylon filter into a sample vial. The vial opening was covered with foil and pierced 3× and allowed to evaporate at ambient. The solids that formed were isolated for evaluation.

### Solubility Determination

Aliquots of various solvents were added to measured amounts of stated materials with agitation (typically sonication) at stated temperatures until complete dissolution was achieved, as judged by visual observation. If dissolution occurred after the addition of the first aliquot, values are reported as “>”. If dissolution did not occur, values are reported as “<”.

### Slurry

Solutions were prepared by adding enough solids to a given solvent so that excess solids were present. The mixture was then agitated in a sealed vial at either ambient or an elevated temperature. After a given amount of time, the solids were isolated for analysis.

### Differential Scanning Calorimetry (DSC)

DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, and the weight was accurately recorded. The pan was then inserted into the DSC cell. A weighed aluminum pan configured as the sample pan was placed on the reference side of the cell. The pan lid was pierced prior to analysis. Samples were analyzed from −30° C. to 250° C. @ 10°/min.

### Dynamic Vapor Sorption/Desorption (DVS)

Automated vapor sorption (VS) data were collected on a Surface Measurement System DVS Intrinsic instrument. Samples were not dried prior to analysis. Sorption and desorption data were collected over a range from 5% to 95% RH at 10% RH increments under a nitrogen purge. The equilibrium criterion used for analysis was less than 0.0100% weight change in 5 minutes with a maximum equilibration time of 3 hours. Data were not corrected for the initial moisture content of the samples.

### Karl Fischer

Coulometric Karl Fischer (KFC) analysis for water determination was performed using a Mettler Toledo DL39 KF titrator. A NIST-traceable water standard (Hydranal Water Standard 1.0) was analyzed to check the operation of the coulometer. A blank titration was carried out prior to sample analyses. The sample was prepared at ambient conditions, where a weighed amount of the sample was dissolved in approximately 1 mL Hydranal-Coulomat AD in a pre-dried vial. The entire solution was added to the KF coulometer through a septum and mixed for 10 seconds. The sample was then titrated by means of a generator electrode, which produces iodine by electrochemical oxidation: 2 I⁺→I₂+2e⁻. Two replicates were obtained to ensure reproducibility.

### Polarized Light Microscopy

Polarized light microscopy was performed using a Motic SMZ-168. Each sample was observed using a 10× objective at 0.75 up to 5.0× magnification with crossed polarizers.

### Proton NMR Spectroscopy

The solution 1H NMR spectra were obtained at Spectral Data Solutions. The specific acquisition parameters are listed on the spectral data sheet provided for each sample in the data section.

### X-ray Powder Diffraction (XRPD)

a. Transmission

XRPD pattern was collected with a PANalytical X'Pert PRO MPD or PANalytical Empyrean diffractometer using an incident beam of Cu radiation produced using a long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening and asymmetry from axial divergence. Diffraction patterns were collected using a scanning position-sensitive detector (X'Celerator) located 240 mm from the specimen and Data Collector software v. 2.2b or 5.5. The data acquisition parameters are listed in the image of each pattern displayed in the Data section of this report. All images have the instrument labeled as X'Pert PRO MPD regardless of the instrument used.

b. Reflection Geometry (Samples in Limited Quantity)

XRPD patterns were collected with a X'Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 5.5. The data acquisition parameters for each pattern are displayed above the image in the Data section of this report including the divergence slit (DS) and the incident-beam SS.

c. Variable Temperature X-Ray Powder Diffraction (VT-XRPD)

XRPD patterns were collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Data were collected and analyzed using Data Collector software v. 2.2b or 5.5. Prior to the analysis, a silicon specimen (NIST SRM 640e) was analyzed to verify the observed position of the Si 111 peak is consistent with the NIST-certified position. A specimen of the sample was packed in a nickel-coated copper well. Antiscatter slits (SS) were used to minimize the background generated by air scattering. Soller slits for the incident and diffracted beams were used to minimize broadening from axial divergence. Diffraction patterns were collected using a scanning position-sensitive detector (X'Celerator) located 240 mm from the sample. The data acquisition parameters for each pattern are displayed above the image in the Data section of this report including the divergence slit (DS) and the incident-beam SS.

An Anton Paar TTK 450 stage was used to collect in-situ XRPD patterns as a function of temperature. The sample was heated with a resistance heater located directly under the sample holder, and the temperature was monitored with a platinum-100 resistance sensor located in the specimen holder. The heater was powered and controlled by an Anton Paar TCU 100 interfaced with Data Collector.

### Example 2

### A. Received Materials

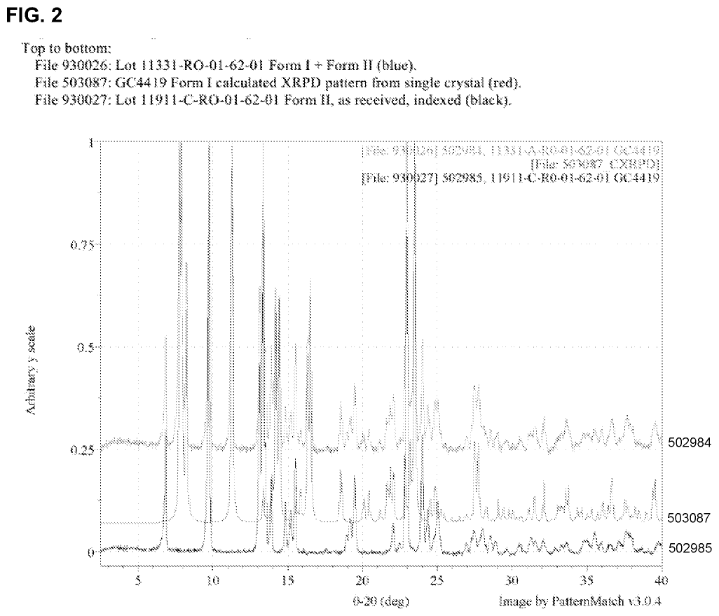

Two lots were received for use. XRPD data was obtained on both (FIG. 2) which are referred to herein as the “Input Materials”. One Input Material was determined to be a dihydrate and the other a mixture of a dihydrate and a monohydrate. The samples were prepared as set forth in Example 3.

### B. Characterization

VT-XRPD experiments on the dihydrate Input Material and the mixture of the dihydrate and monohydrate Input Materials were conducted to investigate the thermal events further. For these experiments, the material is heated, held at specific temperatures, and analyzed by XRPD in situ. Form VIII is observed at −80° C. By 100° C., sample displacement occurred (due to physical expansion of the material) and data acquisition had to be temporarily stopped in order to repack the sample. After resuming the experiment, complete conversion to Form VIII was achieved by 203° C. Form VIII was recovered from the experiment upon cooling and characterized. A mixture of Form VIII and Form XI was obtained near 307° C. in another experiment. In additional hearing experiments, mixtures of Form VI and Form VIII were obtained upon exposure to elevated temperatures up to 205° C. Discoloration, likely associated with thermal decomposition, was evident at 195° C. and above if a nitrogen purge was not used. At 205° C., complete conversion to Form VIII was achieved. Mixtures of Form XI and Form VIII were obtained at 370° C. (under nitrogen).

Form V appears to be a sesqui or dihydrate. Form V was isolated, sometimes as a mixture with other forms, from several evaporative experiments involving water, aqueous IPA, or aqueous acetone mixtures. For example, when the Input Material, which is a mixture of monohydrate and dihydrate, was solubilized in water and then rapidly evaporated, it resulted in crystals of Form V. Continued exposure of Form V to ˜0% RH for 20 days resulted in a disordered mixture of Form V and other solid forms.

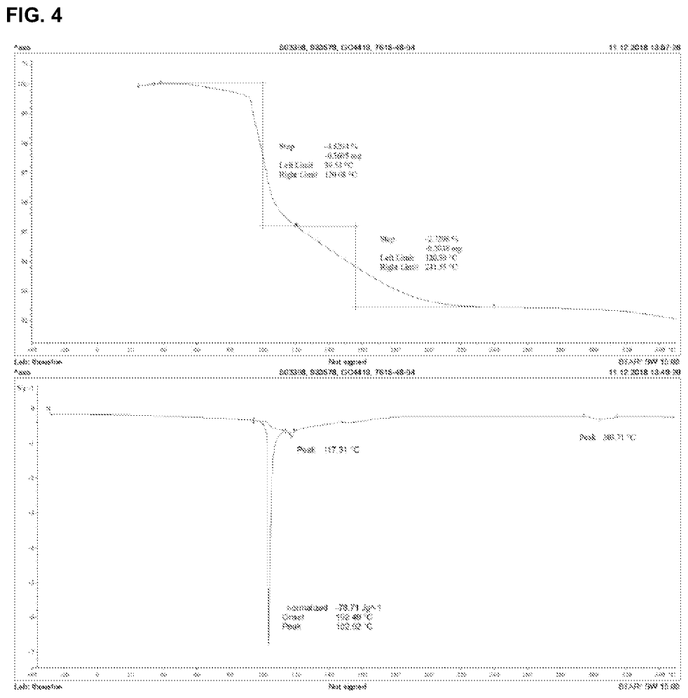

Thermograms of Form V are presented in FIG. 4. A two-step weight loss of 4.8% from 40 to 120° C. and an additional 2.7% from 120 to 242° C. is observed in the TGA. The first step corresponds to a loss of ˜1.5 mol/mol of water. The total two-step loss corresponds to ˜2.2 mol/mol of water. An endotherm with an onset at 102° C. is observed in the DSC. Additional small events are also observed in the DSC above this temperature.

Form VI was obtained by exposing either Input Material to ˜0% RH or elevated temperatures. Based on the conditions used to achieve the material, Form VI is assumed to be anhydrous. No conversion was observed upon stressing at 48% RH for 17 days.

A single crystalline phase of Form VIII was obtained by dehydrating the Input Material dihydrate at ˜205° C. under nitrogen. Mixtures of Form VIII with Form VI were obtained through the dehydration of the dihydrate Input Material at slightly elevated temperatures below 205° C. or at ˜0% RH. Form VIII is not physically stable and can hydrate to the monohydrate.

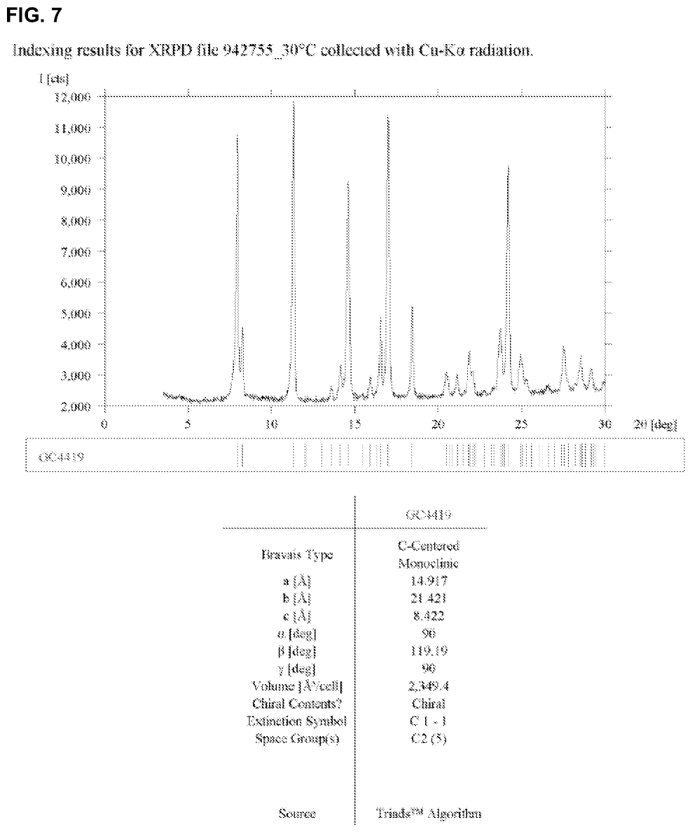

A representative XRPD pattern of Form VIII was indexed (FIG. 7). The indexing volume is ˜16 Å³ per formula unit smaller than Form I monohydrate, consistent with an anhydrous form.

Form VIII is not physically stable and can hydrate to the monohydrate.

Form XI, as mixtures with Form VIII, was obtained by continued exposure of Form VIII to elevated temperatures above ˜300° C. Additional attempts to isolated Based on the conditions used to achieve the material, Form XI is assumed to be anhydrous.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

### Example 3—Sample Preparation

An exemplary method for the synthesis of GC4403 is as follows. GC4419 can be prepared using substantially the same synthesis but substituting the mirror image tetraamine starting material (e.g., mirror image of M40400 depicted below), to arrive at the mirror image stereoisomer of GC4403.

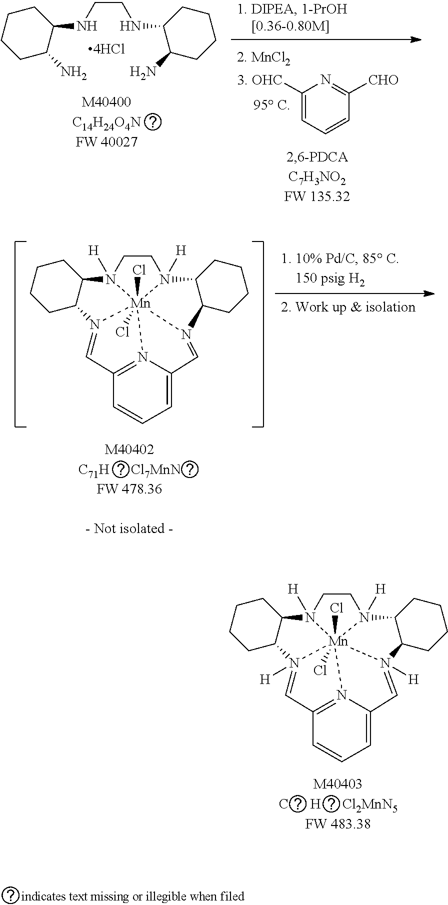

An exemplary synthesis of GC4403 (and thus an exemplary synthesis of GC4419 using the mirror image of M40400 as a starting material) is provided below.

Chemicals, materials and methods: Ultra-dry manganese (II) chloride (99.99%, 42844) and palladium on carbon (10% Pd content, standard, reduced, nominally 50% H₂O content, 38304) were purchased from Alfa-AEsar. N,N″-Diisopropylethylamine (DIPEA, re-distilled, 99.5%, 38,764-9), 1-propanol (99.5+%, HPLC grade, 29,328-8) and filter agent, Celpure® P65 (USP-NF, pharmaceutical grade, 52,523-5) were purchased from Aldrich Chemical Co. 2,6-Pyridinedicarboxaldehyde (2,6-PDCA) was manufactured by ABC Laboratories, Columbia, Missouri. Tetraamine tetrahydrochioride M40400 was manufactured by either of CarboGen Laboratories AG, Aarau, Switzerland, Gateway Chemical Technology, St. Louis, Missouri or ABC Laboratories, Columbia, Missouri. 2-Propanol (99.9%, HPLC grade, A451-4), as well as all other solvents (HPLC-grade unless otherwise indicated) and reagents were purchased from VWR Scientific Products or Fisher Scientific and were of the finest grade available. “Reduced pressure” refers to operations carried out using a rotary evaporator and vacuum provided by a circulating water pump. “In vacuo” refers to high vacuum operations (≤50.5 torr), achieved with the use of an efficient, high capacity vacuum pump and an on-line dry-ice/2-propanol trap (capable of maintaining a temperature of ca. −40° C.). Elemental analysis was performed by Desert Analytics in Tucson, AZ (Mn and Na) or by Atlantic Microlab in Norcross, GA (all others). Reaction progress and product homogeneity was determined by HPLC analysis using a Varian ProStar system coupled to a Waters Symmetry-Shield™ RPS-18, 5 μm (4.6×250 mm) column. Solvent systems A and B consisted of 0.5 M LiCl/0.125 M TBAC in water and 1:4 (v/v) water:acetonitrile (CH₃CN), respectively. Elution was accomplished over a 20-min period using a 95:5 (v/v) A:B solvent mixture, run isocratically at a flow rate of 1.0 mL/min and UV detection at 265 nm. Samples for HPLC analysis were diluted/dissolved using mobile phase to afford typical analyte concentrations ca. 1 mg/mL and ca. 20 μL of this solution was injected.

### Cyclization Stage

An exemplary three-component template cyclization stage is described. Tetraamine hydrochloride M40400 (15.06 g, 37.6 mmol) was suspended in 1-propanol (110 mL, rendered a ca. 0.35 M mixture in M40400), thoroughly blanketed with Ar for 15 minutes, and stirred using a 19×35 mm Teflon blade (overhead stirrer, 700 rpm). DIPEA (26.2 mL, 4 equiv, 150.4 mmol) was added in three portions as a stream to the white suspension, which in less than a minute turned into a nearly colorless light syrup. After 10 min, MnCl₂ (4.78 g, 1 equiv, 38.0 mmol) was added in one portion. Thirty minutes after MnCl₂ addition, a nearly clear faint yellow solution had resulted (depending on the scale and efficiency of Ar purging the color may vary) and 2,6-pyridinedicarboxaldehyde (5.08 g, 1 equiv, 37.6 mmol) was added in one portion. The solution turned yellow-orange and heating commenced immediately, reaching 95° C. within 20 min. One hour later, 95% product conversion was detected by HPLC. Following a total of 4 h at 95±2° C., the brownish solution was cooled to near rt over 30 min. HPLC showed ca. 97% M40402 and the crude mixture was used as such immediately.

### Reduction Stage

An exemplary catalytic hydrogenation of bisimine M40402 is described. Once cool (ca. 30° C.), the mixture resulting from the cyclization stage described above was transferred to a Parr stainless steel reactor vessel for catalytic hydrogenation. The dark mixture was blanketed with Ar for a 2-3 minutes, then 10% Pd/C (3.0 g total, 50% water wet, 10 wt % dry catalyst w/respect to M40400) was carefully added. The reactor was assembled and purged of air by pressurizing/depressurizing with N₂ (0 to 150 to 0 psig) five times. Next, the procedure was repeated three times using H₂ (0 to 150 to 0 psig), and finally the reactor was charged with H₂ (150 psig). The suspension was stirred (700 rpm) and heating commenced immediately. The set temperature, 85° C., was reached within 15 min. The reaction was stirred under pressure overnight for a total of 18 h. At this time, HPLC showed the reaction stream contained ca. 99% M40403. After bringing to rt over 1 h and purging with N₂ (5× as before), the reactor was disassembled and the suspension filtered through a bed of 1-propanol-washed celite (10 g) using a 25-50p fritted funnel. The catalyst/celite bed was washed with 1-propanol (2×20 mL). The yellow solution was evaporated under reduced pressure until an opaque light yellow semi-solid remained (water bath temperature ≤35° C.). This material was stirred in water (700 mL, total solution volume ca. 800 mL) until dissolved (ca. 10 min). The pH of the faint yellow solution was measured as 5.82 and adjusted to 7.78 using 10% aq. NaOH (ca. 0.5 mL). NaCl (210 g, yields a ca. 25% solution in NaCl) was added to the slightly hazy light yellow solution (total volume after NaCl addition ca. 900 mL). After stirring for 20 minutes, the off-white suspension was filtered using a 25-50μ fritted funnel. The cake was sucked dry under reduced pressure for approximately 5 minutes (at this time, no more foam dripped), transferred to a beaker, stirred (700 rpm using a 0.25×1 inch magnetic bar) in 20% aqueous NaCl (75 mL) for 15 min, and filtered as above. This washing/filtering procedure was repeated twice more in exactly the same manner. After the second wash, the left over off-white wet material was dried in vacuo (40° C., 17 h, 0.1 torr, cooled to rt over 1 h). Crude M40403 was isolated (19.06 g, 105% mass yield from M40400) as an off-white solid with a purity >99% by HPLC (This crude product contains ca. 5% NaCl and a water content of 5.8% corresponding to a 1.5 hydrated species). Once cool, the solid was broken down to a free-flowing powder prior to 2-propanol extraction. The solid was magnetically stirred (0.25×2 in bar, 800 rpm) in 200 mL of 2-propanol for 30 min and filtered through a 0.5-in bed of celite (pre-washed with 2-propanol immediately before filtration to avoid any possible water absorption). Water (100 mL) was added to the clear light-yellow filtrate and the mixture was briefly swirled to homogenize. Solvents were then removed under reduced pressure (water bath temperature ≤35° C.)⁸ to afford an off-white solid that was further dried in vacuo (40° C., 18 h, 0.1 torr). M40403 was isolated as an off-white solid (16.63 g, 91% yield from M40400) with a purity >99% by HPLC. The material was broken down to a powder and stored in a freezer at 2-8° C.

### Embodiments

Embodiments of the disclosure herein include, but are not limited to, the following:
Embodiment 1: Form V of GC4419.
Embodiment 2: Form VI of GC4419.
Embodiment 3: Form VIII of GC4419.
Embodiment 4: Form XI of GC4419.
Embodiment 5: Form V of GC4403.
Embodiment 6: Form VI of GC4403.
Embodiment 7: Form VII of GC4403.
Embodiment 8: Form XI of GC4403.

What is claimed is:

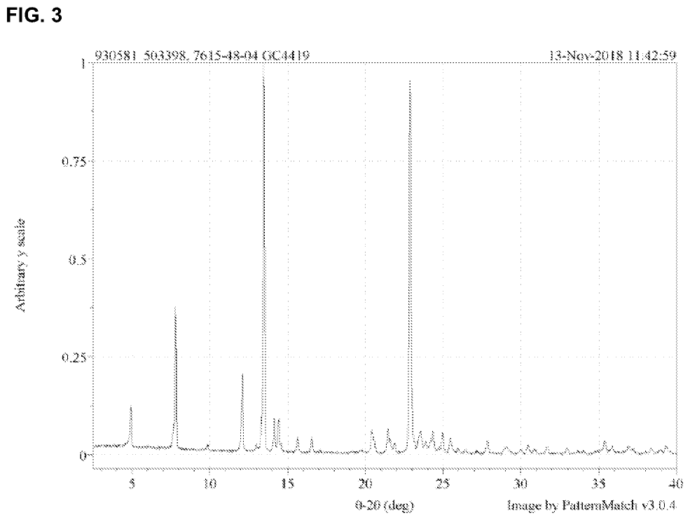

1. A Form V crystalline form of GC4419 according to the following formula: 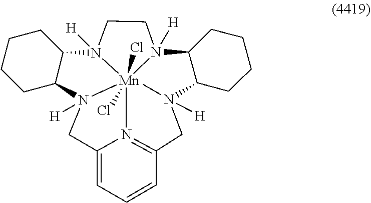
having one or more characteristic XRPD (X-Ray Powder Diffraction) peaks as shown in FIG. 3.

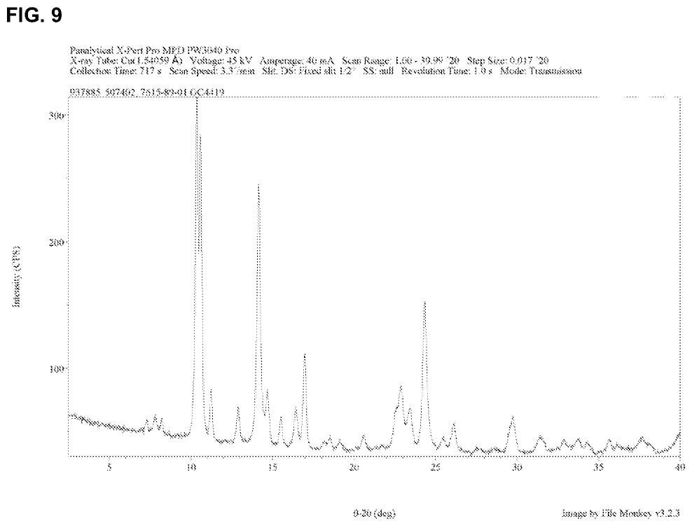

2. A Form VI crystalline form of GC4419 according to the following formula: 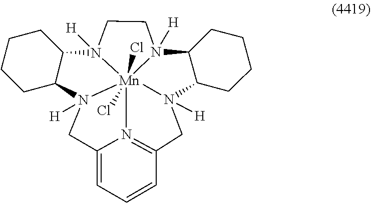
having one or more characteristic XRPD peaks as shown in FIG. 9.

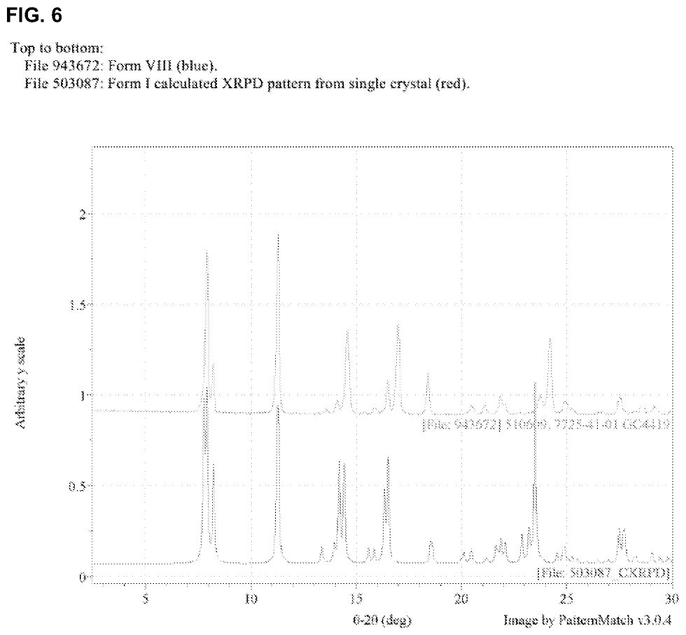

3. A Form VIII crystalline form of GC4419 according to the following formula: 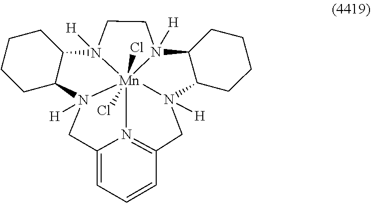
having one or more characteristic XRPD peaks as shown in FIG. 6.

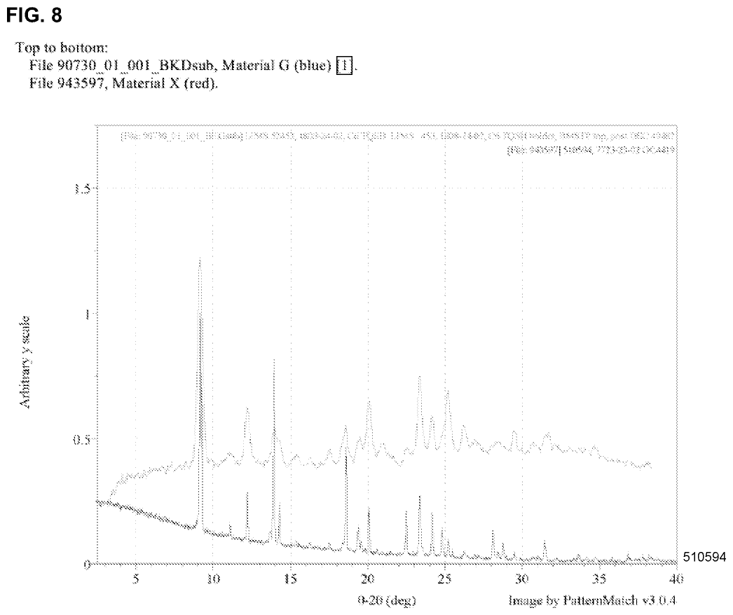

4. A Form XI crystalline form of GC4419 according to the following formula: 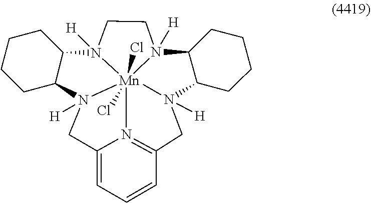
having one or more characteristic XRPD peaks as shown in FIG. 8.

5. A pharmaceutical composition comprising one or more of Form V GC4419, Form VI GC4419, Form VIII GC4419 and Form XI GC4419.

6. A method of treating cancer comprising administering a composition of claim 5 and one or more pharmaceutically acceptable excipients to a patient in need thereof.

7. A method of treating an inflammatory condition comprising administering a composition of claim 5 and one or more pharmaceutically acceptable excipients to a patient in need thereof.

8. The method of claim 7, wherein the inflammatory condition is oral mucositis.
